(19) United States
(12) Patent Application Publication
(10) Pub. No.: US 2018/0161491 A1
(43) Pub. Date: Jun. 14, 2018

(54) Packaging For Safety Needle

(71) Applicant: Becton, Dickinson and Company, Franklin Lakes, NJ (US)

(72) Inventors: Laurie Sanders, Glen Ridge, NJ (US); Edward P. Browka, Oneida, NY (US); Peter Smith, Cary, NC (US); Adam Kristopher Brakoniecki, Hawthorne, NJ (US); Alice Wong, Leonia, NJ (US); Regina Haywood, Franklin Lakes, NJ (US)

(21) Appl. No.: 15/837,018

(22) Filed: Dec. 11, 2017

Related U.S. Application Data
(60) Provisional application No. 62/479,563, filed on Mar. 31, 2017.
(60) Provisional application No. 62/479,507, filed on Mar. 31, 2017.
(60) Provisional application No. 62/433,044, filed on Dec. 12, 2016.

(51) Int. Cl. A61M 5/00; B65D 75/36; B65D 75/58; B65D 75/52

(52) U.S. Cl. CPC A61M 5/002; B65D 75/366; B65D 2203/02; B65D 75/5855; B65D 75/527; B65D 75/367

(57) ABSTRACT

Single and dual packaging for fill needles and safety needles are described herein. Such packaging can include hard packaging or blister packs. Such packaging can include a cavity or recess in a compartment of the packaging to house an activation element of a safety needle device to prevent accidental activation of the safety needle device prior to use by the user.

### CROSS-REFERENCE TO RELATED APPLICATIONS

This application claims priority under 35 U.S.C. § 119(e) to U.S. Provisional Application No. 62/433,044, filed Dec. 12, 2016, U.S. Provisional Application No. 62/479,507, filed Mar. 31, 2017 and U.S. Provisional Application No. 62/479,563, filed Mar. 31, 2017, the disclosures of which are incorporated herein by reference in their entirety.

### TECHNICAL FIELD

The present disclosure generally relates to packaging for medical devices, and more particularly to dual packaging for passive safety needles requiring rotation of a tether or housing for passive activation.

### BACKGROUND

Clean or sterile articles particularly useful for medical applications are packaged to preserve their sterility. The packaging for these articles is intended to provide a barrier to prevent microorganisms from entering inside the packaging to contaminate its contents. In most instances, the packaging is opened immediately prior to using the article, such as with a blister pack housing a syringe or a needle, so as to minimize the time period in which the article is exposed to unsterile conditions.

Traditionally, practitioners that fill and inject syringes can use a one or two-needle technique. In the one-needle technique, the practitioner fills the syringe from a container (e.g. vial) having a liquid contained therein, and uses the same needle for injection. In the two-needle technique, the practitioner fills the syringe with a first needle, but replaces the needle with a new needle prior to injecting.

Both the one-needle technique and the two-needle technique offer certain advantages and disadvantages. For example, the one-needle technique is convenient because the practitioner does not have to change needles between filling and injection, but the needle can become contaminated between filling and injection. The two-needle technique allows for specialized needles that are optimized for filling and injection, but is more cumbersome for the practitioner.

Accordingly, there is a need for alternative packaging systems for providing needles to a user in a sterile condition. There is also a need to provide packaging to prevent unintended activation for passive safety needles that requiring rotation of a tether or housing for passive activation.

### SUMMARY

One aspect of the present disclosure pertains to a packaging system comprising a single-use passive safety needle device and a hard package. The safety needle device includes a protruding activation element. The hard package includes a closed distal end, an open proximal end, and a compartment extending between the closed distal end and the open proximal end. The compartment includes a recess to nest the protruding activation element of the safety needle device. The hard package includes a removable portion sealed against the open proximal end, the compartment and the removable portion defining a sealed region, the safety needle device being disposed within the sealed region.

In one or more embodiments, the safety needle device is a passive safety needle or an active safety needle.

In one or more embodiments, the removable portion comprises a pull tab.

Another aspect of the present disclosure pertains to a packaging system comprising a needle, a safety needle device, a first hard package and a second hard package. The needle has a distal end and a proximal end and the safety needle device has a protruding activation element. The first hard package includes a first compartment, the second hard package includes a second compartment extending between the closed distal end and the open proximal end, and the first hard package can be attached to the second hard package. The second compartment has a recess to nest the protruding activation element of the safety needle device. The first hard package is attached to the second hard package. A first removable portion is sealed against the first compartment, the first compartment and the first removable portion defining a first sealed region, and a second removable portion is sealed against the second compartment, the second compartment and the second removable portion defining a second sealed region.

In one or more embodiments, the first needle is a blunt fill needle. In one or more embodiments, the safety needle device is a passive safety needle or an active safety needle.

In one or more embodiments, the first hard package is attached to the second hard package via press fitting, an adhesive bond, a solvent bond, a ring connector, a snap fit, a C-clip snap, heat staking or ultrasonic welding.

In one or more embodiments, the first hard package has a perforated attachment to the second hard package.

In one or more embodiments, the first removable portion comprises a first pull tab and the second removable portion comprises a second pull tab.

Another aspect of the present disclosure pertains to a packaging system including a needle, a safety needle device and a blister package. The first needle has a distal end and a proximal end and the second needle has a protruding activation element. The blister package includes a first cavity and a second cavity having a recess to nest the protruding activation element of the safety needle device, the first and second cavities sealed against a backing, the first cavity and the backing defining a first sealed region, and the second cavity and the backing defining a second sealed region.

In one or more embodiments, the entire first needle is disposed within the first sealed region. In one or more embodiments, the entire second needle is disposed within the second sealed region.

In one or more embodiments, the first needle is a blunt fill needle. In one or more embodiments, the safety needle device is a passive safety needle or an active safety needle.

In one or more embodiments, the packaging system also includes a cap disposed about the distal end of the needle.

### BRIEF DESCRIPTION OF THE DRAWINGS

FIG. 1 illustrates a single packaging for a safety needle device;

FIG. 2 illustrates a section view of the single packaging of FIG. 1 having a recess in a compartment of the packaging to nest a protruding activation element of a safety needle device;

FIG. 3 illustrates a dual packaging with two pull tabs;

FIG. 4 illustrates a hard package having two removable portions with two pull tab;

FIG. 5 illustrates a blister pack containing a blunt fill needle and a safety needle.

### DETAILED DESCRIPTION

Before describing several exemplary embodiments of the present disclosure, it is to be understood that the embodiments of the present disclosure are not limited to the details of construction or process steps set forth in the following description. The embodiments of the present disclosure are capable of other embodiments and of being practiced or being carried out in various ways.

With respect to terms used in this disclosure, the following definitions are provided.

As used herein, the use of “a,” “an,” and “the” includes the singular and plural.

Reference to “needle” includes needles that are suitable for filling and/or injecting liquids into or out of a syringe. In this disclosure, a convention is followed wherein the portion of a needle closest to the practitioner operating the needle is termed “proximal” and the portion of the needle toward the patient (for injection) or vial containing liquid (for filling) and farthest from the practitioner is termed “distal.” In various embodiments, the needles described herein can be blunt fill needles, safety needles and/or conventional needles.

As used herein, a “fill needle” refers to a needle that is suitable to fill a syringe but may not be suitable for injection. For example, a fill needle may be a blunt needle that is not suitable to penetrate a patient's skin.

As used herein, a “safety needle” refers to a needle suitable for injection that includes one or more features to prevent needle stick injuries. In one or more embodiments, a safety needle includes a sheath that covers the distal end of the needle. As used herein, an “active safety needle” refers to a safety needle with a user-operated activation mechanism to cover the distal end of the needle after a patient has been injected. As used herein, a “passive safety needle” refers to a safety needle with a passive activation mechanism that automatically covers the distal end of the needle after a patient has been injected.

Any suitable needle devices comprising a safety feature may be used in conjunction with the packaging disclosed herein. Exemplary safety needle devices include, but are not limited to, those described in commonly owned, U.S. Patent Application Nos. 62/433,294, 62/433,350, 62/479,507, 62/533,786, the disclosures of which are incorporated herein by reference in their entireties. Types of safety features vary in structure and mechanics but in general, safety needle devices have a fill state and an inject state.

Reference to “syringe” includes syringes that are indicated for use with needles, nozzle, tubing, or for use in flush systems. As used herein, the term “syringe” refers to a simple pump-like device consisting of a plunger rod that fits tightly in a barrel or tube. The plunger rod can be pulled or pushed along inside the barrel, allowing the syringe to take in and expel a liquid or gas through an opening at the open end of the barrel. The open end of the syringe may be fitted with a needle, nozzle, or tubing to help direct the flow of fluid into and out of the barrel. The syringe may be sterile or unsterile, depending upon the needs of the technician.

As used herein, the terms “package” or “packaging” includes any material used to wrap or protect a good or product, such as a syringe or a needle. Packaging can be rigid or flexible. Packaging includes, but is not limited to, medical packaging, pharmaceutical packaging, and child-resistant packaging. Medical and pharmaceutical packaging can include blister packs or hard packages.

As used herein, the terms “blister package” or “blister pack” includes several types of pre-formed packaging used for consumer goods, foods, pharmaceuticals, medical devices, etc. The primary component of a blister pack is a cavity or pocket made from a formable web, usually a thermoformed plastic. The formable web can be rigid or flexible. The cavity or pocket is large enough to contain the good which is housed in the blister package. Depending on the application, a blister pack may have a backing of thermoformable material and a lidding seal of aluminum foil, paper, Tyvek®, plastic, or other medical grade materials. Blister packs can also be hinged, clamshell containers, that can include a rigid backing, such as paperboard. Blister packages can provide barrier protection from microorganisms and other contaminants, and can provide a certain degree of tamper resistance. Within the many options that blister packaging provides, the blister pack must protect the product contained inside while still possessing the characteristic capable of making automated processing possible.

Blister packs are commonly used as unit-dose packaging for pharmaceutical tablets, capsules, or lozenges. The pharmaceutical product and its blister pack act together to serve as an integral unit. The blister pack protects the pharmaceutical product from outside influences that would otherwise render it useless while allowing the manufacturer of the pharmaceutical product to package it using form-fill-seal equipment. The form-fill-seal process involves creating the blister pack from rolls of flat sheet or film, filling with the pharmaceutical product, such as a drug tablet, and closing (sealing). This type of blister pack is sometimes referred to as push-through-packs because the consumer can push the good (e.g. drug tablet) through the backing. With pharmaceutical blister packs, manufacturers must be concerned with the moisture vapor transmission rate of the blister pack because many pharmaceutical products degrade and lose their efficacy through hydrolysis. Additionally, the blister pack must provide a barrier to oxygen in order to prevent degradation of the pharmaceutical product through oxidation. In one or more embodiments, the blister pack is a push-through-pack.

Blister packages that contain medical devices, such as a syringe, differ from pharmaceutical blister packs because medical blister packs are not push-through packages. Instead, the thermoformed base web is made of a thicker plastic and cannot be collapsed, thus forming a solid backing. The lidding film provides a peel-open feature that can be peeled open using two-hands, such as, e.g. the knuckle-roll-peel technique. The lidding film of a medical blister pack is generally porous to allow sterilization. Often, medical blister packs are made of Tyvek® or a similar medical grade material that is breathable and permeable to gases, but is not permeable to microorganisms. The lidding film can also be made of medical grade paper or a completely non-permeable or non-breathable film. In instances where a non-breathable film is used, sterilization is through radiation (e.g. electron beams (E-beam)). In one or more embodiments, the blister pack is a medical blister pack.

Blister packs can be created via thermoforming or cold forming. In the case of thermoforming, a plastic film or sheet is unwound from a reel and guided through a pre-heating station on the blister line. The temperature of the pre-heating plates is such that the plastic will soften and become pliable. The warm plastic then arrives in a forming station where a large pressure forms the blister cavity into a negative mold. The mold is cooled such that the plastic becomes firm again and maintains its shape when removed from the mold.

In the case of cold forming, an aluminum based-laminate film is simply pressed into a mold by means of a stamp. The aluminum elongates and maintains the formed shape. The use of aluminum offers a complete barrier for water and oxygen. However, cold form blister packs take longer to produce compared to thermoforming. Cold form blister packs are also not transparent, which can lead to consumers not complying with pharmaceutical therapies.

The thermoformable backing of the medical blister pack is generally comprised of a flexible thermoformable plastic film. The film is often multi-layered. The primary component is regularly a layer of approximately 15-30% Nylon, while the remaining layers can comprise substances including, but not limited to, polyethylene. The sealant layer can comprise, among others, ethyl vinyl acetate (EVA).

The lidding film of a medical blister pack can be made from plastic, aluminum, or medical grade papers that are permeable to gases for sterilization but are impermeable to microorganisms. Most commonly, Tyvek® is used as a lidding material for medical blister packs.

Blister packaging can also include the skin pack, where a paperboard or other backing material and product are covered with a thin sheet of transparent plastic. The backing generally has a heat-seal coating. The plastic film is softened by heat and draped over the product on the backing. Vacuum is sometimes used to assist in a snug fit. Immediately after forming the blister, the blister is transported to a vacuum sealing station where a vacuum is pulled and the blister is sealed shut, providing the snug fit. The plastic film bonds to the heat-seal coating on the paperboard or other backing. In one or more embodiments, the blister pack is a vacuum sealed thermoformed blister pack.

Blister packs can be sealed in a variety of ways including, but not limited to, heat-sealing and cold sealing. Lidding materials can have a heat-seal coating applied to them; the lidding is then sealed to the backing using heat, which activates the coating. Blister packs can also be sealed using a cold seal process, which uses a combination of a pressure sensitive fold-over blister card and a transparent blister; the blister is trapped between two pieces of board that are bonded together under pressure without using any heat. Additionally, blister packs can be sealed by orienting multiple layers of film properly in order to make a seal.

As used herein, the term “hard package” or the like includes packaging having a compartment with one or more openings that can be covered to create a seal. In one or more embodiments, the hard package includes one or more components made of a rigid material such as a rigid polymeric material. Examples of rigid polymeric materials include, but are not limited to, polyester, polycarbonate, polyethylene, polystyrene or polypropylene, or combinations or co-polymers thereof. In one or more embodiments, a hard package can thermoformed or molded, such as by injection molding. The techniques described above for blister packs can be applied to the rigid portions of hard packages and/or to removable portions of hard packages.

As used herein, the term “microorganism” refers to a microbe or organism that is unicellular or lives in a colony of cellular organisms. Microorganisms are very diverse; they include, but are not limited to bacteria, fungi, archaea, and protozoans.

Tyvek® is a synthetic material consisting of flashspun high-density polyethylene fibers (i.e. a spunbound olefin fiber). The material is lightweight and strong, and is resistant to tearing but can be cut with scissors or a knife. Water vapor and other gases can pass through Tyvek® as the material is highly breathable, but, at the same time, the material is impermeable to liquid water and microorganisms.

As used herein, the term “sterilization” refers to a means of eliminating or killing microorganisms present on a surface, contained in a fluid or in a compound such as biological culture media in order to achieve asepsis or a sterile microbial environment. Sterilization can be achieved by applying heat, chemicals, irradiation/radiation, high pressure, filtration, or combinations thereof. Chemical sterilization includes sterilization with gases such as ethylene oxide, hydrogen peroxide gas, and ozone, liquids such as chlorine bleach, iodine, glutaraldehyde and formaldehyde, ortho-phthaladehyde (OPA), hydrogen peroxide, peracetic acid, sodium hydroxide, silver, and cobalt. Radiation sterilization involves the use of radiation such as electron beams (E-beam), x-rays, gamma rays, or subatomic particles.

As used herein, the term “knuckle-roll-peel technique” refers to the process whereby a technician, such as a doctor or nurse, opens a package to release the product contained therein. With a knuckle-roll motion, the outer packaging material is peeled apart using two hands, and the inner product is released.

Various embodiments of the present disclosure provide dual packaging systems containing two needles. In one or more embodiments, this dual packaging can help to improve work flow and efficiency for users of the two-needle technique by removing the need to remember to get two needles instead of one. In one or more embodiments, this dual packaging can also be helpful for clinicians who traditionally use a one-needle technique to fill and inject, as such practitioners may not be used to getting a separate packaged component. In one or more embodiments, this dual packaging can also help to drive compliance in clinical settings where managers want clinicians to use a two-needle technique but the clinicians would prefer to use the more convenient one-needle technique. In one or more embodiments, dual packaging can be beneficial because it helps to prevent a user from injecting a patient with a device in the fill state either accidentally or purposefully. For passive safety, injection with a device in a fill state could prevent the safety from activating. In one or more embodiments, providing two needles allows a user to perform injection with a second needle that has not been dulled, recapped, or undergone risk of touch contamination. In one or more embodiments, the two needles include a fill needle (e.g. blunt fill needle) and a needle for injection (e.g. a safety needle). In other embodiments, one or both of the needles is a conventional needle.

One or more embodiments of the present disclosure relate to a single compartment or dual compartment packaging system having a hard package.

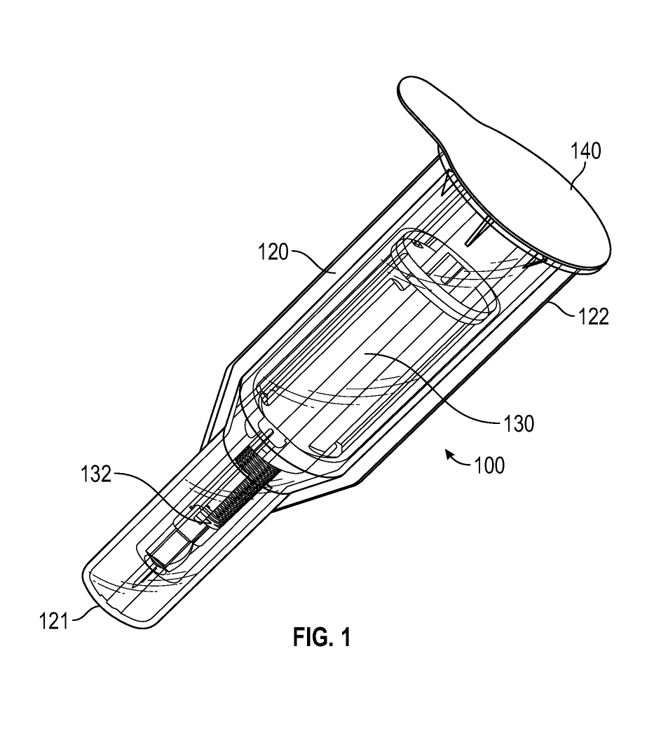

FIG. 1 illustrates an exemplary embodiment of a single compartment packaging system 100. Compartment 120 that can house a safety needle device 130 having a protruding activation element 132. As shown in FIG. 1, the compartment 120 can be molded in a single piece, such as by injection molding.

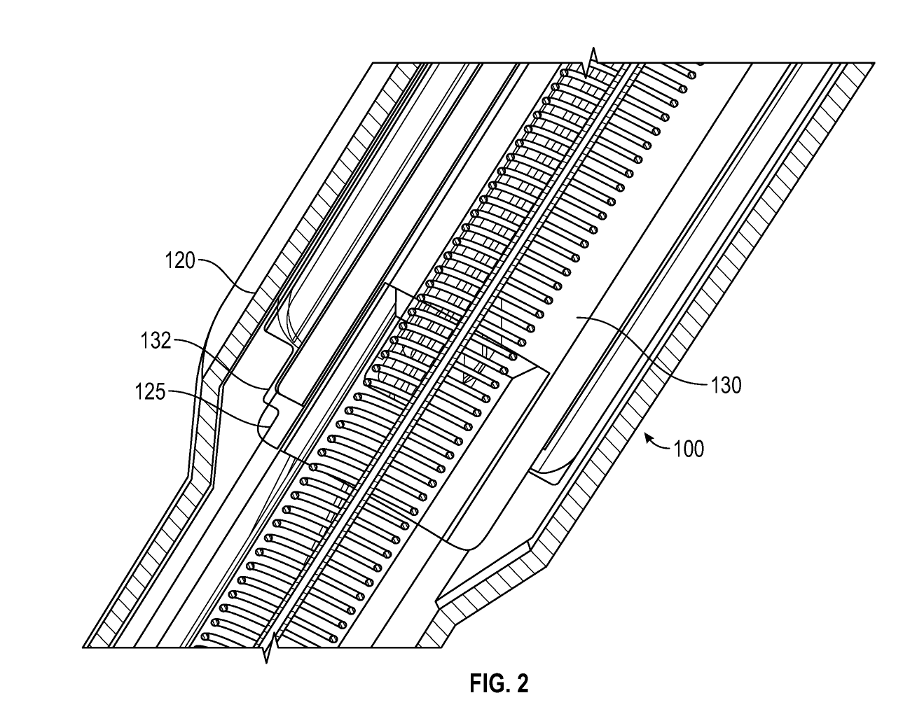

As shown in FIG. 1, the single compartment packaging system 100 may include a hard package having a closed distal end 121, an open proximal end 122, and a compartment 120 extending between the closed distal end 121 and the open proximal end 122. A removable seal 140 disposed against the open proximal end 122. Compartment 120 and removable seal 140 define a sealed region in which the safety needle device is disposed. Removable seal 140 can be a pull tab. As shown in FIG. 2, a premature activation prevention element 125 in the compartment can nest the protruding activation element 132 of the safety needle device 130. In one or more embodiments, the premature activation prevention element 125 is a recess in the compartment. In one or more embodiments, the premature activation prevention element 125 interdigitates with a tether or a retractable sleeve of the safety needle device.

Safety needle device is a passive safety needle or an active safety needle.

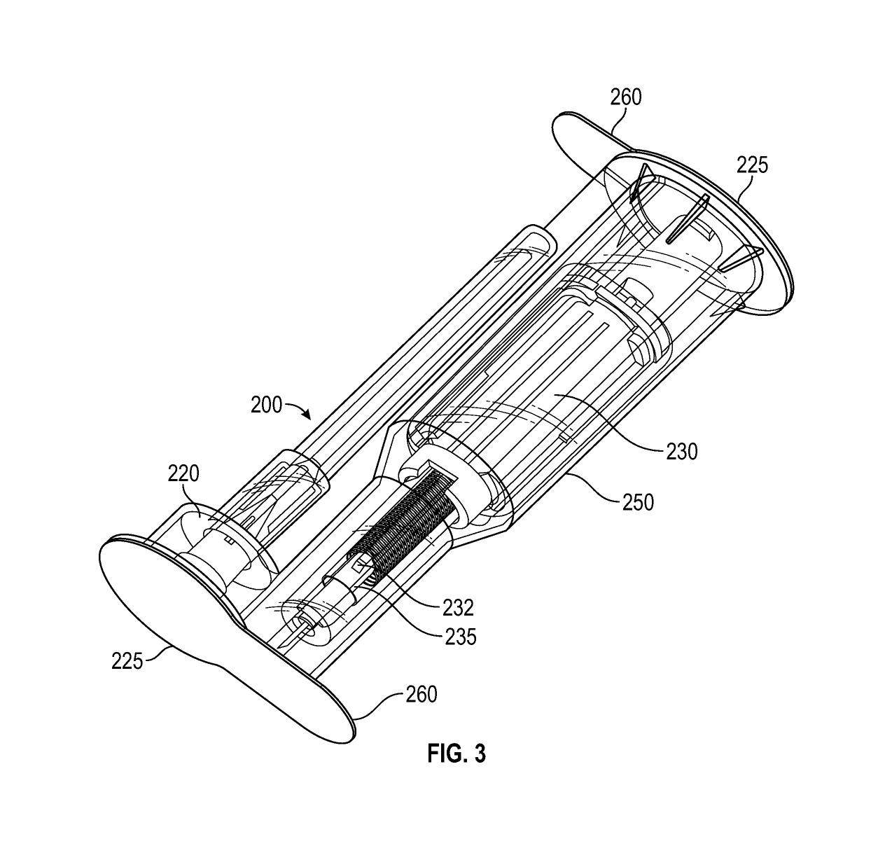

FIG. 3 illustrates an exemplary embodiment of a dual packaging system 200. The packing system has a first compartment 220 that can house a first needle and a second compartment 250 that can house a safety needle device 230. As shown in FIG. 3, the first compartment 220 and second compartment 250 can be molded in a single piece, such as by injection molding.

The first compartment 220 has an opening that is covered by a first removable seal 225. The first removable seal 225 is sealed against the first compartment 220, with the first compartment 220 and the first removable seal 225 defining a first sealed region. The first removable seal 225 can include a pull tab for a user to grab in order to remove the first removable seal 225 to access the first compartment 220.

In one or more embodiments, the first removable seal 225 can include graphics, symbols, diagrams, words or other instructions to indicate that it is to be opened first. For example, the first removable seal 225 can include the number “1”. The first removable portion may also include graphics, symbols, diagrams, words or other instructions to indicate the intended use of the needle stored in the first compartment 220. In one or more embodiments, the first removable seal 225 can include graphics, symbols, diagrams, words or other instructions to indicate that it is to be opened first.

The second compartment 250 has an opening that is covered by a second removable seal 255. Second compartment 250 includes a premature activation prevention element 235 in the compartment can nest the protruding activation element 232 of the safety needle device 230. In one or more embodiments, the premature activation prevention element 235 is a recess in the compartment. In one or more embodiments, the premature activation prevention element 235 interdigitates with a tether or a retractable sleeve of the safety needle device 230. The second removable seal 255 is sealed against the second compartment 250, with the second compartment 250 and the second removable seal 255 defining a second sealed region. The second removable seal 255 can include a second pull tab 260 for a user to grab in order to remove the second removable seal 255 and access second compartment 250.

The second removable seal 255 can include graphics, symbols, diagrams, words or other instructions to indicate that it is to be opened second. For example, the second removable seal 255 can include the number “2”. The second removable portion may also include graphics, symbols, diagrams, words or other instructions to indicate the intended use of the needle stored in the second compartment 250. For example, if the needle stored in the second compartment 250 is to be used for injecting a patient, the second removable seal 255 can include a graphic showing a person with a syringe. Furthermore, if the needle stored in the second compartment 250 is a single-use needle device that locks after use, the second removable seal 255 can also include a lock symbol.

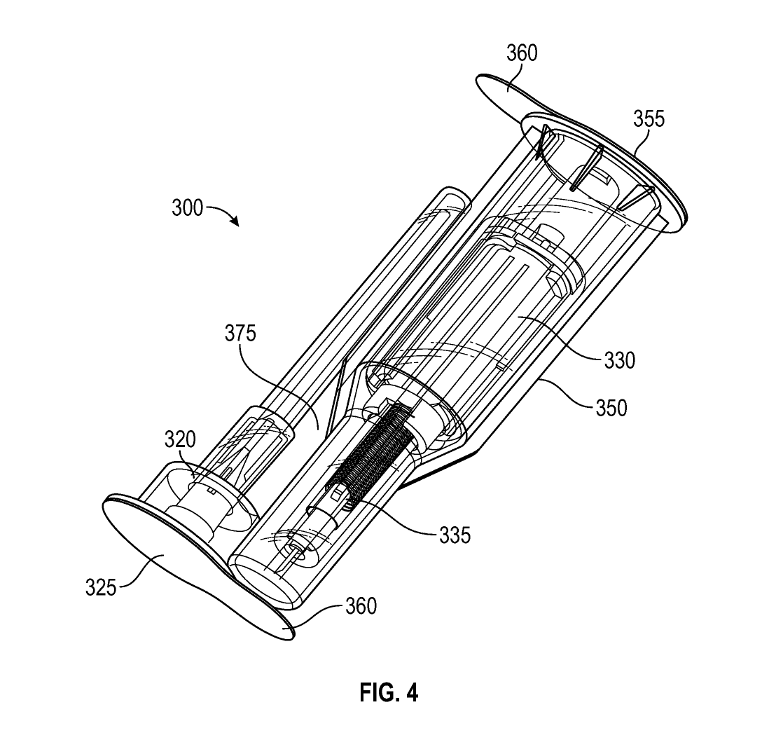

FIG. 4 illustrates an exemplary embodiment of a dual packaging system 300. The packing system has a first compartment 320 that can house a first needle and a second compartment 350 that can house a safety needle device 330. As shown in FIG. 4, the first compartment 320 and second compartment 350 can be molded separately and joined by an attachment 375. In one or more embodiments, the first compartment 320 can be attached to the second compartment 350 via press fitting, an adhesive bond, a solvent bond, a ring connector, a snap fit, a C-clip snap, heat staking or ultrasonic welding. In one or more embodiments, the first compartment 320 can be attached to the second compartment 350 by a perforated or temporary attachment.

In one or more embodiments, the first compartment 320 and the second compartment 350 are composed of different materials or have different colors. For example, in one or more embodiments the first compartment 320 can be colored to indicate a specific use (e.g. red to indicate a blunt fill needle) and the second compartment 350 can be clear, semi-transparent or have a different color indicating a specific use. In other embodiments, both the first compartment 320 and the second compartment 350 are clear or semi-transparent, or are the same color.

The first compartment 320 has an opening that is covered by a removable portion 325. The first removable seal 325 is sealed against the first compartment 320, with the first compartment 320 and the first removable seal 325 defining a first sealed region. The first removable seal 325 can include a first pull tab for a user to grab in order to remove the first removable seal 325 to access the first compartment 320.

The second compartment 350 has an opening that is covered by a second removable seal 355. The second removable seal 355 is sealed against the second compartment 350, with the second compartment 350 and the second removable seal 355 defining a second sealed region. The second removable seal 355 can include a second pull tab 360 for a user to grab in order to remove the second removable seal 355 and access the second compartment 350.

Second compartment 350 includes a premature activation prevention element 335 in the compartment can nest the protruding activation element 332 of the safety needle device 330. In one or more embodiments, the premature activation prevention element 335 is a recess in the compartment. In one or more embodiments, the premature activation prevention element 335 interdigitates with a tether or a retractable sleeve of the safety needle device 330. The second removable seal 355 is sealed against the second compartment 350, with the second compartment 350 and the second removable seal 355 defining a second sealed region. The second removable seal 355 can include a second pull tab 360 for a user to grab in order to remove the second removable seal 355 and access second compartment 350.

The first removable seal 325 and second removable seal 355 can include any of the features described above for the first removable seal 325 and second removable seal 355, such as graphics, symbols, diagrams, words or other instructions to indicate the order of opening compartments or the intended use of the needles stored in the compartments.

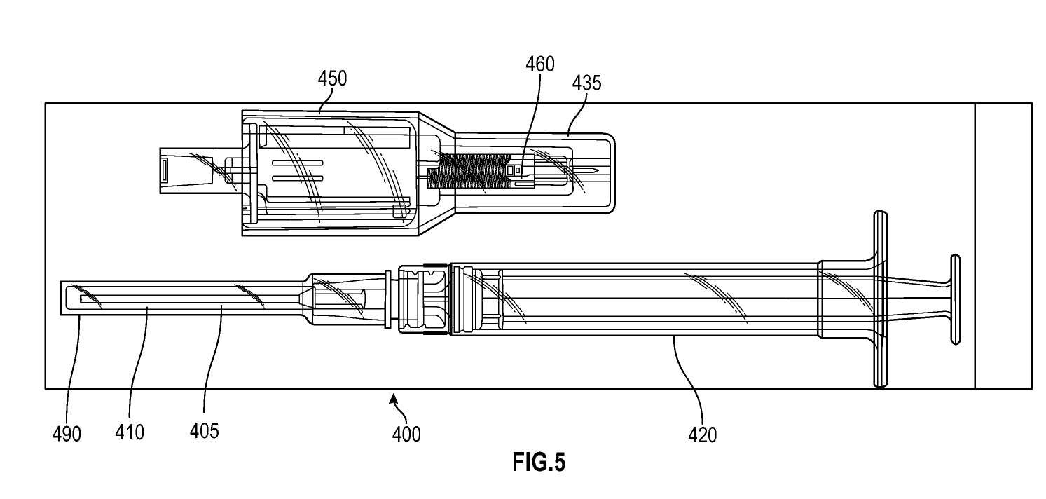

One or more embodiments of the present disclosure relate to a dual packaging system having a blister package. An exemplary embodiment of a dual packaging system 400 is shown in FIG. 5. A first cavity 420 houses at least a portion of a needle 405, and a second cavity 450 houses at least a portion of a safety needle device 435. In one or more embodiments, first cavity 420 houses a syringe and pre-attached blunt fill needle. Safety needle device 435 having a protruding activation element 436. In one or more embodiments, needle 405 and safety needle device 435 can be packed side by side in dual blister packaging system 400.

In one or more embodiments, second cavity 450 having a recess 460 to nest the protruding activation element of the safety needle device. The distal portion 410 of the needle 405 can be covered with a more rigid material than the proximal portion 415 of the first needle. The distal portion 410 of the needle 405 can be covered by a cap 490. Similarly, the distal portion 440 of the safety needle device 435 can be covered with a rigid material. The distal portion 445 of the safety needle device 435 can be covered by a cap 490. A backing 485 can provide a seal against the first cavity 420 and the second cavity 450. The blister package can also include a peel tab 495 to open the blister package. In one or more embodiments, direction of peel is on the right hand side of the packaging to reduce the chance of contamination. In another embodiment, direction of peel is on the left hand side of the packaging.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A packaging system comprising:
a safety needle device having a protruding activation element; and
a hard package having a closed distal end, an open proximal end, and a compartment extending between the closed distal end and the open proximal end;
a premature activation prevention element in the compartment to nest the protruding activation element of the safety needle device; and
a removable seal disposed against the open proximal end, the compartment and the removable seal defining a sealed region, the safety needle device being disposed within the sealed region.

2. The packaging system of claim 1, wherein the safety needle device is a passive safety needle or an active safety needle.

3. The packaging system of claim 1, wherein the premature activation prevention element is a recess.

4. The packaging system of claim 1, wherein the removable seal includes a pull tab.

5. A packaging system comprising:
a needle having a distal end and a proximal end;
a safety needle device having a protruding activation element;
a first hard package comprising a first compartment;
a second hard package having a closed distal end, an open proximal end, and a second compartment extending between the closed distal end and the open proximal end;
a recess in the second compartment to nest the protruding activation element of the safety needle device, wherein the first hard package is attached to the second hard package;
a first removable seal sealed against the first compartment, the first compartment and the first removable seal defining a first sealed region, the needle being disposed within the first sealed region; and
a second removable seal sealed against the second compartment, the second compartment and the second removable seal defining a second sealed region, the safety needle device being disposed within the second sealed region.

6. The packaging system of claim 5, wherein the needle is a blunt fill needle.

7. The packaging system of claim 5, wherein the safety needle device is a passive safety needle.

8. The packaging system of claim 5, wherein the safety needle device is an active safety needle.

9. The packaging system of claim 5, wherein the first hard package is attached to the second hard package via press fitting, an adhesive bond, a solvent bond, a ring connector, a snap fit, a C-clip snap, heat staking or ultrasonic welding.

10. The packaging system of claim 5, wherein the first hard package has a perforated attachment to the second hard package.

11. The packaging system of claim 5, wherein the first removable portion comprises a first pull tab and the second removable portion comprises a second pull tab.

12. The packaging system of claim 11, wherein the first removable seal includes graphics, symbols, diagrams, words or instructions to indicate that the first removable seal is to be opened first.

13. The packaging system of claim 11, wherein the second removable seal includes graphics, symbols, diagrams, words or instructions to indicate that the first removable seal is to be opened second.

14. The packaging system of claim 11, wherein the first hard package has a first color and the second hard package has a second color.

15. A packaging system comprising:
a needle having a distal end and a proximal end;
a safety needle device having a protruding activation element; and
a blister package having a first cavity and a second cavity having a premature activation prevention element to nest the protruding activation element of the safety needle device, the first and second cavities sealed against a backing, the first cavity and the backing defining a first sealed region, the second cavity and the backing defining a second sealed region, the distal end of the needle being disposed within the first sealed region and the distal end of a second needle being disposed within the second sealed region.

16. The packaging system of claim 15, wherein the needle is a blunt fill needle.

17. The packaging system of claim 15, wherein the safety needle device is a passive safety needle or an active safety needle.

18. The packaging system of claim 15, wherein the activation prevention element is a recess.

19. The packaging system of claim 15, further comprising a cap disposed about the distal end of the needle.

20. The packaging system of claim 15, wherein the blister package includes a peel tab.
